(19) United States
(12) Plant Patent Application Publication
(10) Pub. No.: US 2004/0268453 P1
(43) Pub. Date: Dec. 30, 2004

(54) Botanical/ commercial classification senecio cruentus x Senecio heritieri/Cineraria plant verietal denomination cv. Sunsenerabu'

(76) Inventor: Kiyoshi Miyazaki, Hikone (JP)

Correspondence Address: CHRISTIE, PARKER & HALE, LLP, PO BOX 7068, PASADENA, CA, 91109-7068, US

(21) Appl. No.: 10/611,353

(22) Filed: Jun. 30, 2003

(51) Int. Cl. A01H 5/00

(52) U.S. Cl. PLT/263

(57) ABSTRACT

Disclosed herein is a Senecio plant named ‘Sunsenerabu’; being of a high height and dome-shaped plant with abundant branching. Petiole length is longer than in Senecio L. It has large inflorescence cluster. Inflorescences are single flowered and have small obscure marginal variegation. The color of the ray floret is light violet and the disc floret is strong violet. Blooming term is longer than Senecio cruentus. Flower buds grow one after another from axil.

### BOTANICAL/COMMERCIAL CLASSIFICATION

Senecio cruentus×Senecio heritieri.

### VARIETAL DENOMINATION

cv. ‘Sunsenerabu’

### BACKGROUND OF THE VARIETY

The present invention relates to a new and distinct variety of Senecio genus plant named ‘Sunsenerabu’. ‘Sunsenerabu’ is a distinct and unique variety, which is a dome-shaped plant of height with abundant branching, light violet inflorescences, and a long blooming term.

There are many varieties of Senecio cruentus cultivated in the world, including varieties with inflorescences of a single color of white, pink, red, blue or violet. Some varieties have marginal variegation with off color parts.

The female parent used in the crossing of ‘Sunsenerabu’ is a clone of our own breeding line, Senecio cruentus, ‘8S-84e’ (not patented in the United States), which is a compact, dome-shaped plant, 16 cm in height. The stems are thick, 8.0 mm in diameter, with no anthocyanin coloration. The leaf is in a serrated heart form with moderate yellow green coloration. The leaf size is medium, 12.0 cm long, and 12.5 cm wide. The inflorescence is single flowered and has white ray florets with white disc florets having no marginal variegation. ‘8S-84e’ has some scent.

The pollen parent used in the crossing of ‘Sunsenerabu’ is our breeding line, Senecio heritieri (an unnamed plant; not patented or sold in the United States), which was first introduced from England. Senecio heritieri is a high, dome-shaped plant, 26 cm in height with abundant branching. Stems are 5.1 mm in diameter, with no anthocyanin coloration. The leaf is in a serrated heart form and light yellowish green in coloration. The leaf size is small, 5.5 cm long and 6.3 cm wide. The inflorescence is single flowered, having strong purple ray florets with vague white parts and strong reddish purple disk florets. Senecio heritieri has no scent.

### Progress

The controlled crossing of a plant of Senecio cruentus ‘8S-84e’ and a plant of Senecio heritieri (unnamed) was conducted at Omori-cho, Yokaichi-shi, Shiga, Japan in February, 1999. Seedlings from this crossing have been grown since September, 1999. Four strains were selected in January 2000 in view of flower color and earliness of bloom. After multiplication by tissue culture, the botanical characteristics of the selected new strains were tested in pots starting in September 2000, using the parent varieties, ‘Sunsenebu’ (U.S. Plant Pat. No. 12,104, issued 25 Sep. 2001), and ‘Miss Yokohama’ (not patented in the United States) for comparison.

One seedling was propagated by a cutting at Yokaich-shi, shiga-ken, Japan. The new variety reproduces true to type in successive generations of asexual reproduction. This new variety of Senecio plant was named ‘Sunsenerabu’ (Senecio cruentus×Senecio heritieri).

The parent plants, Senecio cruentus, ‘8S-84e’, and Senecio heritieri (unnamed), are presently maintained at Shiga-ken, Japan.

In the following descriptions, the color-coding is in accordance with the Horticultural Colour Chart of The Royal Horticultural Society, London, England (R.H.S).

The botanical characteristics of the female parent plant Senecio cruentus, ‘8S-84e’, used in the crossing of ‘Sunsenerabu’ are as follows.

Plant:

Growth habit.—Dwarf compact.

Height.—Approximately 16 cm.

Stem:

Thickness.—Approximately 8.0 mm.

Color.—Moderate yellowish green (Near R.H.S. 139C).

Anthocyanin coloration.—Absent.

Branching.—Fair.

Pubescence.—Sparse.

Length of third internode below flag leaf.—Approximately 0.5 cm.

Leaf:

Whole shape.—Heart form with a swollen basal part, which is notched where the basal part of the blade attaches to the petiole.

Depth of concavity of leaf margin.—Medium.

Type of convexity.—Acute.

Apex shape.—Obtuse.

Base shape.—Cordate.

Degree of Undulation.—Fair.

Length.—Approximately 12.0 cm.

Width.—Approximately 12.5 cm.

Diameter of petiole.—Approximately 5.0 mm.

Length of petiole.—Approximately 5.5 cm.

Color of upper surface.—Moderate yellow green (Near R.H.S. 137C).

Color of reverse surface.—Grayish yellow green (Near R.H.S. 138B).

Anthocyanin coloration of reverse surface.—Absent.

Pubescence of upper surface.—Present.

Pubescence of reverse surface.—Dense.

Color of pubescence of reverse surface.—White.

Flower (single flowered):

Shape of flower cluster.—Flat.

Diameter of flower cluster.—Approximately 20 cm.

Height of flower cluster.—Approximately 8 cm.

Transected shape of corolla.—Flat.

Diameter of flower.—Approximately 5.5 cm.

Size of disk flower.—Approximately 1.2 cm.

Color of petal.—White (Near R.H.S. 155D).

Marginal variegation.—Absent.

Color of disk flower.—White (Near R.H.S. 155D).

Petal length.—Approximately 2.1 cm.

Petal width.—Approximately 1.0 cm.

Shape of petal.—Elliptical.

Lengthwise warp of petal.—Flat.

Concavity of patal tip.—Present.

Shape of petal tip.—Rounded.

Number of ray flower.—Approximately 13.

Number of disk flower.—Approximately 105.

Diameter of pedicel of the first flower.—Approximately 1.6 mm.

Length of pedicel of the first flower.—Approximately 2.4 cm.

Number of flowers per a flower cluster.—Approximately 75.

Scent.—Present.

Calyx:

Degree of concavity.—Flat.

Degree of recurvature.—Approximately 2.2 mm.

Color.—Moderate yellow green (Near R.H.S. 139C).

Anthocyanin coloration.—Absent.

Pistil:

Color.—Light yellow (Near R.H.S. 11B).

Number.—1.

Type.—Style branches truncate (i.e., the top of the style is separated into two and the shape of the top is truncated).

Stamen:

Color.—Brilliant yellow (Near R.H.S. 12B).

Type.—5 anthers are connate, with separated filaments.

Blooming period.—January (Sowing in August).

Hardiness:

Cold.—Good.

Rain.—Good.

Heat.—Good.

Resistance:

Disease.—Good.

Insect.—Good.

The botanical characteristics of the male parent plant Senecio heritieri used in the crossing of ‘Sunsenerabu’ are as follows.

Plant:

Growth habit.—Dwarf compact.

Height.—Approximately 26 cm.

Stem:

Thickness.—Approximately 5.1 mm.

Color.—Very pale green (Near R.H.S. 128D).

Anthocyanin coloration.—Absent.

Branching.—Abundunt.

Type of primary lateral shoot.—Branch from every node.

Pubescence.—Dense.

Length of third internode below flag leaf.—Approximately 0.8 cm.

Leaf:

Whole shape.—Heart form with a swollen basal part, which is notched where the basal part of the blade attaches to the petiole.

Depth of concavity of leaf margin.—Medium.

Type of convexity.—Acute.

Apex shape.—Obtuse.

5Base shape.—Cordate.

Degree of Undulation.—Weak.

Length.—Approximately 5.5 cm.

Width.—Approximately 6.3 cm.

Diameter of petiole.—Approximately 4.2 mm.

Length of petiole.—Approximately 7.5 cm.

Color of upper surface.—Light yellowish green (near R.H.S. 136D).

Color of reverse surface.—Very pale green (near R.H.S.128D).

Anthocyanin coloration of reverse surface.—Absent.

Pubescence of upper surface.—Dense.

Pubescence of reverse surface.—Dense.

Color of pubescence of reverse surface.—White.

Flower (single flowered):

Shape of flower cluster.—Uneven.

Diameter of flower cluster.—Approximately 18 cm.

Height of flower cluster.—Approximately 15 cm.

Transected shape of corolla.—Flat.

Diameter of flower.—Approximately 4.8 cm.

Size of disk flower.—Approximately 0.9 cm.

Color of petal.—Strong purple (near R.H.S.81B).

Marginal variegation.—Present.

Diameter of off color part.—Approximately 1.3 cm.

Border of marginal variegation.—Vague.

Color of disk flower.—Strong reddish purple (Near R.H.S. No.72A).

Petal length.—Approximately 2.2 cm.

Petal width.—Approximately 0.4 cm.

Shape of petal.—Rectangular.

Lengthwise warp of petal.—Flat.

Concavity of patal tip.—Present.

Shape of petal tip.—Acute.

Number of ray flower.—Approximately 13.

Number of disk flower.—Approximately 110.

Diameter of pedicel of the first flower.—Approximately 1.2 mm.

Length of pedicel of the first flower.—Approximately 2.5 cm.

Number of flowers per flower cluster.—Approximately 35.

Scent.—Absent.

Calyx:

Degree of concavity.—Flat.

Degree of recurvature.—Approximately 3.1 mm.

Color.—Very pale green (Near R.H.S. 128D).

Anthocyanin coloration.—Present.

Pistil:

Color.—Strong reddish purple (Near R.H.S. 72A).

Number.—1.

Type.—Style branches truncate (i.e., the top of the style is separated into two and the shape of the top is truncated).

Stamen:

Color.—Strong reddish purple (Near R.H.S. 72A).

Type.—5 anthers are connate, with separated filaments.

Blooming period.—End of January (Sowing in August).

Hardiness:

Cold.—Good.

Rain.—Good.

Heat.—Good.

Resistance:

Disease.—Good.

Insect.—Good.

The botanical characteristics of a similar variety ‘Sunsenebu’ (Senecio cruentus×Senecio heritieri) which was patented in the United States, used for examination as a comparison variety are as follows.

Plant:

Growth habit.—Semi-erect.

Height.—Approximately 33 cm.

Stem:

Thickness.—Approximately 4.5 mm.

Color.—Very pale green (Near R.H.S. 147C).

Anthocyanin coloration.—Present unevenly.

Degree of anthocyanin coloration.—Light.

Branching.—Abundant, i.e., approximately 9 to 11 branches.

Type of primary lateral shoot.—Branch from every node.

Pubescence.—Sparse.

Length of third internode below flag leaf.—approximately 1.4 cm.

Leaf:

Whole shape.—Heart form with a swollen basal part, which is notched where the leaf attaches to the petiole.

Depth of concavity of leaf margin.—Approximately 7 mm on average.

Type of convexity.—Acute.

Apex shape.—Acute.

Base shape.—Cordate.

Degree of Undulation.—Weak.

Length.—Approximately 6.7 cm.

Width.—Approximately 8.0 cm.

Diameter of petiole.—Approximately 3.1 mm.

Length of petiole.—Approximately 7.4 cm.

Color of upper surface.—Moderate yellow green (Near R.H.S. 137C).

Color of reverse surface.—Moderate yellow green (Near R.H.S. 138C).

Anthocyanin coloration of reverse surface.—Present.

Degree of antocyanin coloration.—Light.

Pubescence of upper surface.—Dense.

Pubescence of reverse surface.—Dense.

Color of pubescence of reverse surface.—White.

Flower (single flowered):

Shape of flower cluster.—Uneven.

Diameter of flower cluster.—Approximately 36 cm.

Height of flower cluster.—Approximately 18 cm.

Transected shape of corolla.—Flat.

Diameter of flower.—Approximately 7.7 cm.

Size of disk flower.—Approximately 1.3 cm.

Color of petal.—Vivid violet (Near R.H.S. 89C).

Marginal variegation.—Absent.

Color of disk flower.—Deep violet (Near R.H.S. 93A).

Petal length.—Approximately 3.2 cm.

Petal width.—Approximately 0.8 cm.

Shape of petal.—Rectangular.

Lengthwise warp of petal.—Flat.

Concavity of patal tip.—Present.

Shape of petal tip.—Acute.

Number of ray flower.—Approximately 13-15.

Number of disk flower.—Approximately 135.

Diameter of pedicel of the first flower.—Approximately 1.0 mm.

Length of pedicel of the first flower.—Approximately 5.5 cm.

Number of flowers per a flower cluster.—Approximately 65.

Scent.—Present.

Calyx:

Degree of concavity.—Moderate.

Degree of recurvature.—Approximately 1.0 mm.

Color.—Very pale green (Near R.H.S. 130D).

Anthocyanin coloration.—Present.

Pistil:

Color.—Deep violet (Near R.H.S. 93A).

Number.—1.

Type.—Style branches truncate (i.e., the top of the style is separated into two and the shape of the top is truncated).

Stamen:

Color.—Deep violet (Near R.H.S. 93A).

Type.—5 anthers are connate, with separated filaments.

Blooming period.—Start at the beginning of December (cutting in July).

Hardiness:

Cold.—Good.

Rain.—Good.

Heat.—Good.

Resistance:

Disease.—Good.

Insect.—Good.

The botanical characteristics of a similar variety ‘Miss Yokohama’, used for examination as a comparison variety are as follows.

Plant:

Growth habit.—Dwarf compact.

Height.—Approximately 19 cm.

Stem:

Thickness.—Approximately 5.4 mm.

Color.—Moderate yellow green (Near R.H.S. 139C).

Anthocyanin coloration.—Present.

Degree of anthocyanin coloration.—Medium.

Branching.—Fair.

Pubescence.—Dense.

Length of third internode below flag leaf.—Approximately 0.5 cm.

Leaf:

Whole shape.—Heart form with a swollen basal part, which is notched where the basal part of the leaf attaches to the petiole.

Depth of concavity of leaf margin.—Medium.

Type of convexity.—Acute.

Size of wing (swollen basal part).—Large.

Apex shape.—Obtuse.

Base shape.—Cordate.

Degree of Undulation.—Weak.

Length.—Approximately 17.5 cm.

Width.—Approximately 13.0 cm.

Diameter of petiole.—Approximately 5.2 mm.

Length of petiole.—Approximately 7.7 cm.

Color of upper surface.—Moderate yellow green (Near R.H.S. 137C).

Color of reverse surface.—Moderate yellow green (Near R.H.S. 138C).

Anthocyanin coloration of reverse surface.—Present.

Degree of anthocyanin coloration.—Medium.

Pubescence of upper surface.—Dense.

Pubescence of reverse surface.—Dense.

Color of pubescence of reverse surface.—White.

Flower (single flowered):

Shape of flower cluster.—Rounded.

Diameter of flower cluster.—Approximately 23 cm.

Height of flower cluster.—Approximately 17 cm.

Transected shape of corolla.—Flat.

Diameter of flower.—Approximately 4.2 cm.

Size of disk flower.—Approximately 1.1 cm.

Color of petal.—Vivid violet (Near R.H.S. 96A).

Marginal variegation.—Present.

Diameter of off color part.—Approximately 2.5 cm.

Border of marginal variegation.—Fairy clear.

Color of disk flower.—Deep violet (Near R.H.S. 93A).

Petal length.—Approximately 1.6 cm.

Petal width.—Approximately 0.8 cm.

Shape of petal.—Generally rectangular.

Lengthwise warp of petal.—Convex.

Concavity of patal tip.—Present.

Shape of petal tip.—Rounded.

Number of ray flower.—Approximately 13.

Number of disk flower.—Approximately 86.

Diameter of pedicel of the first flower.—Approximately 1.7 mm.

Length of pedicel of the first flower.—Approximately 2.7 cm.

Number of flowers per a flower cluster.—Approximately 90.

Scent.—Present.

Calyx:

Degree of concavity.—Flat.

Degree of recurvature.—Approximately 1.2 mm.

Color.—Very pale green (Near R.H.S. 130D).

Anthocyanin coloration.—Absent.

Pistil:

Color.—Deep violet (Near R.H.S. 93A).

Number.—1.

Type.—Style branches truncate (i.e., the top of the style is separated into two and the shape of the top is truncated).

Stamen:

Color.—Deep violet (Near R.H.S. 93A).

Type.—5 anthers are connate, with separated filaments.

Blooming period.—January (Sowing in August).

Hardiness:

Cold.—Good.

Rain.—Good.

Heat.—Good.

Resistance:

Disease.—Good.

Insect.—Good.

### SUMMARY OF THE VARIETY

‘Sunsenerabu’ is a dome-shaped plant of height (average height approximately 39.5 cm in the blooming period). There is a branch from every node and branching is abundant, i.e., approximately 9 to 11 branches. The stem is approximately 4.9 mm in diameter with few pubescences.

The leaves are small, approximately 7.6 cm long, approximately 9.2 cm wide. The leaves are shaped in a serrated heart form with a swollen basal part, which is notched where the basal part of the blade attaches to the petiole. The leaf color is strong yellow green, with no anthocyanin coloration on the reverse side. Pubescence on upper side of leaf is sparse and pubescence on the reverse side is dense. The petiole is approximately 9.2 cm long and approximately 3.2 mm in diameter.

The new variety has large inflorescence clusters, which are high and flat in shape (i.e., the locations of the individual inflorescences forming the cluster are flat). An inflorescence cluster means a gathering of heads in a plant of the new variety. A head is an inflorescence consisting of disc florets and a plurality of ray florets. The inflorescence is single flowered with marginal variegation, which has obscure border. The corolla has a flat transected shape (i.e., when the corolla opens, ray florets are flat when viewed from the side). The inflorescence is approximately 7.4 cm in diameter, while the disc floret is approximately 1.3 cm in diameter. The ray floret is approximately 3.0 cm long and approximately 0.8 cm wide. The color of the ray floret is light violet (near R.H.S. 91A), while the color of the disc florets are violet (near R.H.S. 90B). One inflorescence has approximately 13 ray florets and approximately 149 disc florets. A ray floret has a pistil, but no stamen. A disc floret is tubular and has a pistil and a stamen formed by 5 connate anthers with separated filaments. Approximately 180 inflorescences are in a inflorescence cluster. The degree of recurvature of calyx is approximately 2.0 mm. The calyx is flat and has no anthocyanin coloration. Pedicel is approximately 3.5 cm long and approximately 1.3 mm in diameter. The inflorescences have some scent.

Blooming period is medium and blooming term is long. After cutting in July, inflorescences start to bloom at the end of the January in Omori-cho, Yokaichi-shi, Shiga-ken, Japan. The blooming continues from January to May, if under appropriate control. Around a temperature of 15° C., the individual bloom lasts two weeks. The inflorescence buds grow one after another from the axil. The inflorescence is not self-cleaning because ray florets remain attached in a wilted and dry state as the inflorescence fully matures.

The new variety has moderate cold hardiness and resistance to heat. The new variety does not die at around 0° C., but when frost occurs the cells can be necrosed resulting in the death of the plant. Usually, Senecio cruentus has a tendency of slightly reduced growth in a hot season, whereas the new variety has no problem in growing in the hot season.

The fertility of the new variety is low. Generally, Senecio genus plants have high fertility (i.e., bear many seeds per flower). In contrast, ‘Sunsenerabu’ bears no seed or very few seeds per inflorescence. When no seed is formed, there is a part corresponding to a seed coat, which remains in an immature state, i.e., without embryo and endosperm inside the immature seed coat.

The new variety, ‘Sunsenerabu’, differs from similar varieties, ‘Sunsenebu’ and ‘Miss Yokohama’, of the Senecio genus and from parent varieties Senecio heritieri (unnamed) and ‘8S-84e’ in the following points.

1. ‘Sunsenerabu’ is a high type plant, approximately 39.5 cm in height. ‘Sunsenebu’ is a high type plant, approximately 33 cm in height. ‘Miss Yokohama’ is a compact type plant, approximately 19 cm in height. The pollen parent Senecio heritieri is a semi-compact type plant, approximately 26 cm in height and female plant ‘8S-84e’ is a compact type plant, approximately 16 cm in height.

2. The inflorescence of ‘Sunsenerabu’ has light violet ray florets, strong violet disc florets and obscure marginal variegation. ‘Sunsenebu’ has vivid violet ray florets, deep violet disc florets and no marginal variegation. ‘Miss Yokohama’ has vivid violet ray florets, vivid violet disk florets and no marginal variegation. The female parent ‘8S-84e’ has white ray florets, white disc florets and no marginal variegation. The pollen parent Senecio heritieri (unnamed) has strong purple ray florets, strong reddish purple disk florets and white marginal part.

3. The petiole length of ‘Sunsenerabu’ is longer than that of ‘Sunsenebu’ or ‘Miss Yokohama’.

4. ‘Sunsenerabu’ has less pubescence of stems than that of ‘Miss Yokohama’.

5. The blooming term of ‘Sunsenerabu’ is longer than that of ‘Miss Yokohama’.

6. The fertility of ‘Sunsenerabu’ is lower than that of ‘Miss Yokohama’.

The plant height and flower color are most distinctive characteristics of this new variety, ‘Sunsenerabu’.

### BRIEF DESCRIPTION OF THE DRAWING

FIG. 1 is a photograph giving a partial view of the new variety of Senecio genus plant named ‘Sunsenerabu’ planted in a pot.

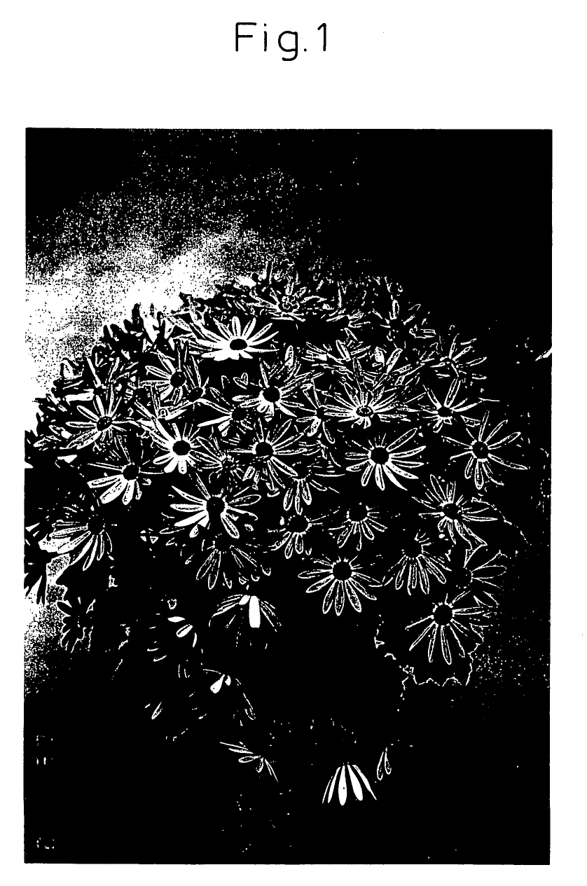

FIG. 2 is a photograph of inflorescences of the new variety of Senecio genus plant named ‘Sunsenerabu’.

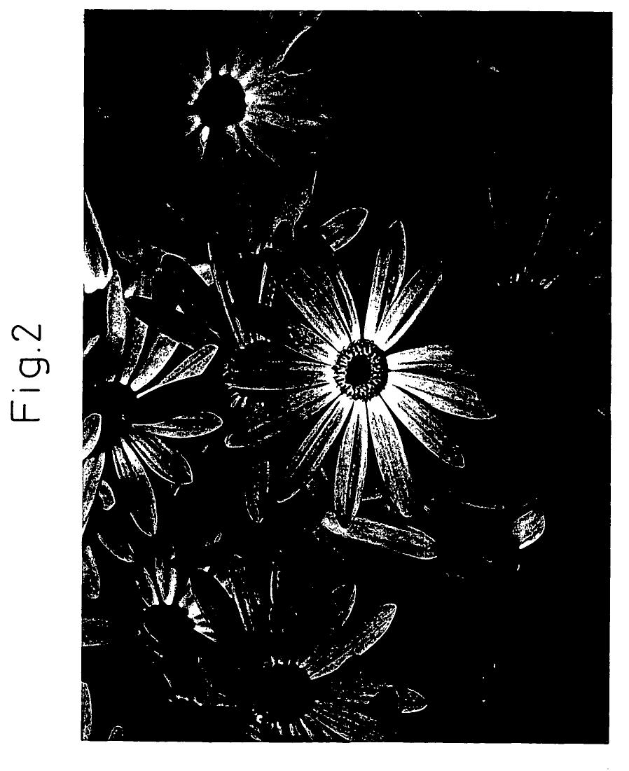

### DESCRIPTION OF THE NEW VARIETY

The botanical characteristics of the new and distinct variety of Senecio plant named ‘Sunsenerabu’ are set forth hereafter. The plants were observed during January 2002 at Yokaichi-shi, Shiga-ken, Japan, planted in 15 cm diameter pots, at an age of approximately 6 months.

Plant:

Growth habit.—Semi-dwarf erect.

Height.—Approximately 39.5 cm.

Spread.—Approximately 45.0 cm.

Main stem length.—Approximately 38.0 cm.

Lateral branches length.—Approximately 20 cm.

Stem:

Thickness.—Approximately 4.9 mm.

Color.—Light yellow green (near R.H.S. 144D).

Anthocyanin coloration.—Absent.

Branching.—Abundant, i.e., approximately 9 to 11 branches.

Type of primary lateral shoot.—Branch from every node.

Pubescence.—Sparse.

Internode length at the middle of main stem.—Approximately 2.1 cm.

Leaf:

Whole shape.—Heart form with a swollen basal part, which is notched where the basal part of the blade attaches to the petiole.

Depth of concavity of leaf margin.—The maximum depth of concavity measured from the average convexity peak height is approximately 5 mm.

Type of convexity.—Acute.

Apex shape.—Acute.

Base shape.—Cordate.

Leaf margin.—Palmately lobed, crenate and weakly undulated.

Length.—Approximately 7.6 cm.

Width.—Approximately 9.2 cm.

Diameter of petiole.—Approximately 3.2 mm.

Length of petiole.—Approximately 9.2 cm.

Color of petiole.—Near R.H.S. 144D.

Color of upper surface.—Strong yellow green (near R.H.S.144A).

Color of reverse surface.—Moderate yellow green (near R.H.S. 138C).

Anthocyanin coloration of reverse surface.—Absent.

Pubescence of upper surface.—Sparse.

Pubescence of reverse surface.—Dense.

Color of pubescence of reverse surface.—White.

Venation.—Pattern Palmate; color near R.H.S. 136D.

Stipules.—None.

Inflorescene (single flowered):

Shape of inflorescence cluster.—Flat.

Diameter of inflorescence cluster.—Approximately 45 cm.

Height of inflorescence cluster.—Approximately 17 cm.

Transected shape of corolla.—Flat.

Diameter of inflorescence.—Approximately 7.4 cm.

Size of disc floret.—Approximately 1.3 cm.

Color of ray floret (upper surface).—Near R.H.S. 91A.

Color of ray floret (lower surface).—Near R.H.S. 91D.

Marginal variegation.—Present.

Size of marginal variegation.—Approximately 1.7 cm.

Border of marginal variegation.—Obscure.

Color of disc floret.—Strong violet (near R.H.S.90B).

Ray floret length.—Approximately 3.0 cm.

Ray floret width.—Approximately 0.8 cm.

Shape of pay floret.—Rectangular.

Lengthwise warp of pray floret.—Flat.

Concavity of tip.—Present.

Shape of ray floret tip.—Acute.

Number of ray flower.—Approximately 13.

Number of disc floret.—Approximately 149.

Diameter of pedicel of the first inflorescence.—1.3 mm.

Length of pedicel of the first inflorescence.—Approximately 3.5 cm.

Pedicel color.—Near R.H.S. 144D.

Number of flowers per a inflorescence cluster.—Approximately 180.

Ray floret margin.—Entire.

Ray floret base shape.—Obtuse.

Ray floret texture.—Smooth, velvety.

Scent.—Present.

Calyx:

Degree of concavity.—Flat.

Degree of recurvature.—Approximately 2.0 mm.

Color.—Strong yellow green (near R.H.S. 143A).

Anthocyanin coloration.—Absent.

Pistil:

Color.—Vivid purple (near R.H.S. 87A).

Number.—1.

Type.—Style branches truncate (i.e., the top of the style is separated into two and the shape of the top is truncated).

Stamen:

Color.—Dark greenish yellow (near R.H.S. 152D).

Type.—5 anthers are connate, with separated filaments.

Amount of pollen.—Scarce.

Pollen color.—Near R.H.S. 21A.

Time to produce.—Approximately 6 months (cutting in July, flower in the next January, in Japan.

Blooming period.—Start at the end of January (cutting in July).

Number of inflorescence per lateral stem.—Approximately 5.

Flowering time.—From January to early May.

Bud:

Hardiness.—Susceptible to damage under 0° C.

Diameter.—Approximately 6 cm.

Length.—Approximately 4 cm.

Shape.—Globose.

Surface.—Pubescent.

Color.—Near R.H.S. 144D.

Lastingness of an individual bloom on the plant.—approximately 10 days.

Hardiness:

Cold.—Good.

Rain.—Good.

Heat.—Good.

Resistance:

Disease.—Good.

Insect.—Good.

The new variety and Senecio cruentus have similar resistance to powdery mildew, leaf spot, aphid, whitefly, and thrips. The new variety, ‘Sunsenerabu’, is a tall type plant and most suitable for flower potting.

This new and distinct variety of Senecio genus plant named ‘Sunsenerabu’ was asexually reproduced by cutting at Omori-cho, Yokaichi-shi, Shiga-ken, Japan and the homogeneity and stability thereof were confirmed.

1. A new and distinct variety of Senecio plant named ‘Sunsenerabu’, substantially as herein illustrated and described.
